(19) United States
(12) Patent Application Publication
(10) Pub. No.: US 2025/0161397 A1
(43) Pub. Date: May 22, 2025

(54) PLANT BASED FORMULATION FOR FAST PAIN RELIEF AND PREPARATION METHOD

(71) Applicant: ARJUNA NATURAL PRIVATE LIMITED, Kerala (IN)

(72) Inventor: Benny ANTONY, Kerala (IN)

(73) Assignee: ARJUNA NATURAL PRIVATE LIMITED, Kerala (IN)

(21) Appl. No.: 18/837,515

(22) PCT Filed: Feb. 10, 2023
(86) PCT No.: PCT/IB2023/051203

§ 371 Date: Aug. 9, 2024

(30) Foreign Application Priority Data
Feb. 16, 2022 (IN) 202241008177

(51) Int. Cl. A61K 36/9066; A61K 9/00; A61K 9/10; A61K 36/324; A61K 47/44; A61P 29/00

(52) U.S. Cl. CPC A61K 36/9066; A61K 9/0053; A61K 9/10; A61K 36/324; A61K 47/44; A61P 29/00

(57) ABSTRACT

Present invention pertains to a plant-based formulation for fast pain relief and method of preparation of said pain relief formulation. Plant-based formulation is made from solid Turmeric extract and solid Boswellia extract loaded into an oil, with a Hydrophilic-lipophilic balance value ranging from 10 to 19. Administering said analgesic and anti-inflammatory formulation hastens the process of relief to a subject experiencing acute musculoskeletal pain.

### FIELD OF THE INVENTION

The present invention relates to a plant-based formulation for fast pain relief and to a method of preparation of said pain relief formulation, more particularly pertaining to a plant-based formulation for Musculoskeletal pain reliefs, made from solid Turmeric extract and solid Boswellia extract loaded into an oil, with a Hydrophilic-lipophilic balance value ranging from 10 to 19. Administering said analgesic and anti-inflammatory formulation hastens the process of relief to a subject experiencing acute musculoskeletal pain.

### BACKGROUND OF THE INVENTION

Musculoskeletal pain refers to pain in the muscles, bones, ligaments, tendons, and nerves. It can be in just one area of the body, such as a leg. It can also be throughout the body, if one has a widespread condition like fibromyalgia. Musculoskeletal pains are of two types, chronic and acute. Acute musculoskeletal pain is sudden-onset pain that affects your muscles, bones (including joints), tendons, ligaments, or nerves. Often, this kind of pain starts immediately after an accident or injury of some type. Conventional treatment with prescribed and over-the-counter drugs such as acetaminophen (paracetamol) or non-steroidal anti-inflammatory drugs (NSAID), such as ibuprofen (e.g., Motrin and Advil) and naproxen (e.g., Aleve and Naprosyn)), and narcotics have remained the mainstay of current treatments. However, these treatments are typically associated with significant adverse side effects (e.g., gastrointestinal, cardiovascular, and addiction).

NSAID available in the market primarily targets the enzymes that produce prostaglandins, called cyclo-oxygenase. There are two cyclo-oxygenase enzymes, COX-1 and COX-2. Both enzymes produce prostaglandins that promote inflammation, pain, and fever; however, only COX-1 produces prostaglandins that activate platelets and protect the stomach and intestinal lining. One downside to the use of NSAIDs is that they reduce the secretion of the protective mucus layer in the stomach and can cause clinically apparent adverse gastrointestinal effects such as peptic ulcers and subsequent risk of bleeding and perforation. Curcumin, a compound prominently found in Turmeric rhizome, on the other hand, is known to inhibit the mRNA and protein expression of COX-2, but not COX-1 (Goel A, Boland C R, Chauhan D P. Specific inhibition of cyclooxygenase-2 (COX-2) express by dietary curcumin in HT-29 human colon cancer cells. Cancer Lett 2001; 172:111-118.). Further, it has been shown that curcumin suppresses the expression of 5-LOX, most likely through the down regulation of NF-κB activation. Additionally, curcumin has been shown to bind to the active site of 5-LOX and inhibit its activity.

Lipoxygenases (LOXs) belong to the multigene family of dioxygenases with content of non-heme iron in the active site of the enzyme. Lipoxygenases are categorized as 5-LOX, 8-LOX, 12-LOX and 15-LOX. 5-LOX is expressed by cells primarily involved in regulating inflammation, allergy, and other immune responses, e.g. neutrophils, eosinophils, basophils, monocytes, macrophages, mast cells, dendritic cells, and B-lymphocytes. In an in vitro study conducted on rat lung cytosolic fraction curcumin was shown as a competitive inhibitor of 12/15 LOX (Bezáková L, Košťálová D, Obložinský M, Hoffman P, Pekárová M, Kollárová R, Holková I, Mošovská S, Sturdík E. Inhibition of 12/15 lipoxygenase by curcumin and an extract from Curcuma longa L. CeskaSlov Farm. 2014 February; 63 (1): 26-31. PMID: 24568335.).

Another plant-derived compound that inhibits 5-LOX is a derivative of Boswellic acid, acetyl-11-keto-β-boswellic acid (AKBA). AKBA is said to inhibit the 5-LOX by an enzyme directed, a non-competitive mechanism via binding to a pentacyclic triterpene-selective effector site (Sailer E R, Subramanian L R, Rall B, Hoernlein R F, Ammon H P, Safayhi H. Acetyl-11-keto-β-boswellic acid (AKBA): structure requirements for binding and 5-lipoxygenase inhibitory activity. Br J Pharmacol. 1996 February; 117 (4): 615-8. doi: 10.1111/j.1476-5381.1996.tb15235.x. PMID: 8646405; PMCID: PMC1909340.). Although AKBA shows good inhibition of 5-LOX, no effect on 12/15-LOX is reported. AKBA might also suppress the activity of COX enzymes by direct interactions; AKBA shows reversible inhibition of COX-1, and COX-2 was less efficiently inhibited by AKBA as compared to COX-1 (Siemoneit U, Hofmann B, Kather N, Lamkemeyer T, Madlung J, Franke L, Schneider G, Jauch J, Poeckel D, Werz O. Identification and functional analysis of cyclooxygenase-1 as a molecular target of Boswellic acids. BiochemPharmacol. 2008 Jan. 15; 75 (2): 503-13. doi: 10.1016/j.bcp.2007.09.010. Epub 2007 Sep. 14. PMID: 17945191.).

Although there is enough in vitro evidence of compounds derived from the plant having analgesic property, plant with analgesic properties is seldom considered for the treatment of musculoskeletal pain due to delay in onset of analgesia. There are herbal formulations for pain and inflammation, but onset to pain relief takes loads of time and they cannot come at par with synthetic drugs such as ibuprofen, acetaminophen, or acetylsalicylic acid.

The U.S. Pat. No. 8,147,882 an herbal formulation is disclosed with 12 herbal extracts. The said formulation is studied for its efficacy, in a non-randomized open-label study. Turmeric extract and Boswellia extract used in the said patent have poor bioavailability and nothing in said formulation is addressing the lack of bioavailability of compounds. Hence the formulation will fail to give instant pain relief. Further, there are about twelve herbs used, but the benefits are not specified, and the use of multiple herbs increases risk of allergic reaction. There is no negative or positive control group used in the study and the subjects were supposed to rate the efficacy based on their memory of the use of any other painkiller drugs. Said study parameters used in U.S. Pat. No. 8,147,882 can lead to a lot of bias and uncertainty, because of which the study results cannot be taken as conclusive evidence. The study as disclosed in example 3 fails to show if the formulation has a fast onset to analgesia to a person who experiences musculoskeletal pain.

In the U.S. Pat. No. 9,352,008, an herbal formulation is disclosed with six herbs to be used for the treatment of pain and stiffness. The human study conducted by the said formulation showed a rejection of pain and stiffness within 2 to 4 weeks, but the study does not show whether the formulation has an immediate effect onset of analgesia on a person who experiences musculoskeletal pain. The study sample is too small to say the study result to be conclusive. Turmeric extract used in the said formulation has poor bioavailability and nothing in said formulation is addressing the bioavailability of the compound.

In the U.S. patent application Ser. No. 13/030,267 a formulation is disclosed, said formulation is made from 19 compounds, many of which are plant extracts. The study conducted to show the efficacy in example 3 is more in line with a marketing survey with no substantial data or scientifically relevant questions. The study does not show if the formulation has an immediate effect on set to analgesia to a person who experiences musculoskeletal pain. Nothing in said formulation is addressing the lack of bioavailability of the compound.

In the U.S. patent application Ser. No. 13/911,265 herbal formulation is disclosed for topical application. It has got about six extracts and alpha-lipolic acid. The use of Turmeric extract and Boswellia extract is below its effective dose. Further Turmeric extract rich in curcumin is not recommended for topical use since it will stain the skin. Concerning pain relief, example 2 discloses a human study and in that study, they have shown improvement in musculoskeletal pain only after two weeks.

Each of the above references uses Turmeric or Boswellia extract in their formulation for the treatment of some type of pain, but in every case, the onset of analgesia is achieved only after a few days or even weeks. Meaningful pain relief on the same day has not been reported yet, which could be because of lack of oral bioavailability of compounds or lack of effective compounds in the formulation. The use of multiple herbs can dilute the effect of relevant active compounds. There is no synergy observed in the above-mentioned formulations, just the addition of properties.

Notwithstanding the above, there is a need for a plant-based pain relief formulation that can provide pain relief at the same rate or better than NSAID. Most NSAIDs have some form of side effect, whereas plant-based products come under a low-risk profile. The general belief is that plant-based pain relief formulations are not an alternative to pharmaceutical pain relief products. The present application discloses a formulation and a method that will change that perspective.

### OBJECTIVE OF THE INVENTION

The principal objective of this invention is an oral formulation for pain relief made from plant extracts that will bring on set to analgesia faster.

Another objective is a method to treat musculoskeletal pain rapidly with a plant-based formulation.

Yet another objective is to develop a method to make a pharmaceutically active pain relief formulation with plant parts.

### SUMMARY OF THE INVENTION

This document discloses a plant-based formulation for a quicker reduction in pain and inflammation, so as to provide faster relief from pain to a person in need of, more especially a formulation for Musculoskeletal pain reliefs, made from solid Turmeric extract and solid Boswellia extract. It also discloses a method of preparation of said formulation. In addition, management of pain in a subject experiencing acute musculoskeletal painto hasten the process of pain relief also is disclosed here.

The present invention relates to a plant-based formulation for fast pain relief and to a method of preparation of said pain relief formulation. More especially pertaining to a plant-based formulation for Musculoskeletal pain reliefs, made from solid Turmeric extract and solid Boswellia extract in about 2.8:1 to 3:1 w/w ratio, loaded into an oil, with a Hydrophilic-lipophilic balance value ranging from 10 to 19. It is having Curcuminoids and Acetyl-11-keto-beta-boswellic acid in a ratio of 2:1 to 99:1. By administering said analgesic and anti-inflammatory formulation hastens the process of relief to a subject experiencing acute musculoskeletal pain.

The analgesic formulation disclosed here is a mixture of solid Turmeric extract having Curcuminoidsand solid Boswellia extract having Acetyl-11-keto-beta-boswellic acid. The mixture is loaded into oil, with a Hydrophilic-lipophilic balance value ranging from 10 to 19. In the analgesic formulation Curcuminoids and Acetyl-11-keto-beta-boswellic acid are in a 2:1 to 99:1 ratio. Curcuminoids consist of curcumin, bisdemethoxycurcumin, and demethoxycurcumin and they are derived from Turmeric rhizome and Acetyl-11-keto-beta-boswellic acid is derived from Boswellia resin.

In an embodiment, the analgesic formulation is a mixture of solid Turmeric rhizome and Boswellia extract uniformly dispersed in the oil, wherein the mixture to oil is in 1:10 to 1:1 weight ratio. Further, the mixture of solid Turmeric extract has at least 90% curcuminoids and solid Boswellia extract with at least 10% Acetyl-11-keto-beta-boswellic acid. The Turmeric extract and Boswellia extract are in 1:1-10:1 w/w ratio, and the mixture is loaded into the oil with an HLB value ranging from 10 to 19. Further, the average particle size of Turmeric and Boswellia extract is below 200 micrometres and above 100 nanometres.

In yet another embodiment a method hastening the onset of analgesia in a subject experiencing acute musculoskeletal pain is disclosed. Administering to the subject in need requires an effective amount of a combination of solid Turmeric extract and solid Boswellia extract, loaded in oil, with a Hydrophilic-lipophilic balance value ranging from 10 to 19. More precisely, administering from about 10 to 25 mg/Kg bodyweight of the combination, wherein the combination comprises of curcuminoids and Acetyl-11-keto-beta-boswellic acid in 30:1 to 10:1 w/w ratio to experience an onset to analgesia is 30 to 210 minutes.

In yet another embodiment, a method to make the pain relief formulation is disclosed. The method of preparation of a plant-based formulation for fast pain relief by enhancing oral bioavailability is comprising the steps of: a) mixing solid Turmeric extract and solid Boswellia extract in about 2.8:1 to 3:1 w/w ratio; b) adding the mixture was added to edible oil and mixed well to get a high viscose blend; and c) passing the resultant blend through a bead mill a number of time sresulting in a yellow coloured liquid with a viscosity in the range of 270 to 320 mP·s.

The mixture of solid Turmeric extract and solid Boswellia extract is blended with at least 50% oil to get a blend. The mixture is then subjected to milling till the mixture is uniformly dispersed in the oil. Further milling the composition till the particle size of the mixture is below 200 micrometres and above 100 nanometres. Anti-oxidants are dosed at 2000 ppm of the said edible oil as preservatives which are a combination of rosemary and green tea extract. Other features of the disclosure will be apparent from the following detailed description.

### DETAILED DESCRIPTION OF THE INVENTION

The present invention discloses a plant-based formulation especially made from a mixture of solid Turmeric extract and solid Boswellia extract loaded into an oil, with a Hydrophilic-lipophilic balance value ranging from 10 to 19 for fast pain relief as well as a method of preparation of said pain relief formulation. The Curcuminoids present in the solid Turmeric extract and Acetyl-11-keto-beta-boswellic acid present in the solid Boswellia extract are in 2:1 to 99:1 ratio. By administrating said analgesic plant-based formulation to a subject experiencing acute musculoskeletal pain hastens the process of pain relief. Each novel feature is disclosed through a number of embodiments. Through these embodiments, a composition, a formulation, and product along with a process of production and method of use are disclosed. The embodiments have to be understood in their broadest sense. The illustrations also disclose the efficacy of said formulations over known counterparts through various in vivo and in vitro studies. The illustrations for the invention are meant for a better understanding of the invention for a person skilled in the art and do not intend to narrow the scope of any subject matter claimed.

The present invention relates to a formulation and method for treatment and management of pain in a subject in need of. Unlike another herbal extract with poor efficacy, the formulation disclosed here is more effective in providing meaningful and also faster pain relief.

Curcuminoids as stated in this application refer to any composition or solution or extract comprised of Curcumin, demethoxycurcumin, and bisdemethoxycurcumin at least 50% of its weight. Curcuminoids may include, solvent extract of Turmeric rhizome, oleoresin derived from Turmeric rhizome, synthetic curcumin and its analogies, Turmeric powder with enriched curcumin or a combination thereof.

Acetyl-11-keto-β-boswellic acid (AKBA) may also be referred to as Acetyl-keto-β-boswellic acid or Acetyl-keto-boswellic acid. AKBA could be an isolated pure form of AKBA or enriched extract of Boswellia, in synthetic form, derived from Boswellia (serrata) resin, or a combination thereof.

As used here, “pain” refers to acute and chronic musculoskeletal pain, including pain caused by physical Blunt trauma, inflammation, back pain, toothache, headache, menstrual cramp, sore throat, fever, and rheumatic pain such as joint pain, arthritis, ankylosing spondylitis, and pain related to systemic connective tissue disorder, and cancer.

In an embodiment analgesic formulation is disclosed, the formulation is a mixture of curcuminoids and AKBA, the mixture is loaded into oil, with a Hydrophilic-lipophilic balance value ranging from 10 to 19. Combination of curcuminoids and AKBA for the analgesic formulation is in 2:1 to 99:1 w/w. The most preferred combination of curcuminoids and AKBA for an analgesic activity is 10:1 to 30:1 w/w. “Loading” or “loaded” refer to uniform mixing of solid compounds into an immiscible liquid, which could be achieved by physical force applied to the mixture to form a uniform dispersant. The oil to be used is an edible oil selected from a group consisting but not limited to sesame oil, coconut oil, palm oil, olive oil, sunflower oil, mustard oil, lemongrass oil, bran oil, groundnut oil, lavender oil, flaxseed oil, grape seed oil, almond oil, avocado oil, soyabean oil, walnut oil, cashew oil, canola oil, corn oil, safflower oil, cottonseed oil, ramtil oil, niger oil, hemp oil, Turmeric oil, ginger oil, animal fat, fish oil, medium-chain triglycerides, saturated fatty acids, unsaturated fatty acid, or combination thereof. The mixture to the oil is in 1:99 to 3:2 w/w ratio, the mixture is uniformly dispersed in oil; a more preferred blend of the mixture to oil will be 1:10 to 1:1, 1:5 to 1:1, and 40% to 60% mixture by weight loaded into the oil. The mixture referred to here is the combination of solid curcuminoids and AKBA.

In a preferred embodiment, the formulation is a mixture of solid Turmeric extract and solid Boswellia extract, and the mixture is loaded into an oil base. The Turmeric extract is enriched with curcuminoids with at least 80% curcuminoids and up to 98% curcuminoids w/w. Curcuminoids consist of curcumin, bisdemethoxycurcumin, and demethoxycurcumin. The solid Boswellia extract with enriched Boswellia acids and at least 10% of the extract is AKBA w/w. The preferred oil for the formulation is vegetable oil, the vegetable oil is selected from the group consisting of but not limited to sesame oil, coconut oil, palm oil, olive oil, sunflower oil, mustard oil, lemongrass oil, bran oil, groundnut oil, lavender oil, flaxseed oil, grape seed oil, almond oil, avocado oil, soyabean oil, walnut oil, cashew oil, canola oil, corn oil, safflower oil, cottonseed oil, ramtil oil, niger oil, hemp oil, turmeric oil, ginger oil, medium-chain triglycerides derived from plants or combination thereof. The mixture to the oil is in 1:99 to 3:2 w/w ratio, the mixture is uniformly dispersed in oil; a more preferred blend of the mixture to oil will be 1:10 to 1:1, 1:5 to 1:1, and 40% to 60% mixture by weight loaded into the oil.

In a preferred embodiment, the formulation is a mixture of solid Turmeric extract and solid Boswellia extract, and the mixture is loaded into an oil base. The Turmeric extract is enriched with curcuminoids with at least 80% curcuminoids and up to 98% curcuminoids w/w. Curcuminoids consist of curcumin, bisdemethoxycurcumin, and demethoxycurcumin. The solid Boswellia extract with enriched Boswellia acids and at least 10% of the extract is AKBA w/w. The Turmeric extract and Boswellia extract are in about 1:1 to 3:1 w/w ratio, more preferably 2.8:1 to 3:1. The preferred oil for the formulation is omega-3 rich fish oil or a combination of omega-3 rich vegetable oil and fish oil. The mixture to the oil is in 1:99 to 1:1 w/w ratio, the mixture is uniformly dispersed in oil. The mixture to the oil is in 1:99 to 3:2 w/w ratio, the mixture is uniformly dispersed in oil; a more preferred blend of the mixture to oil will be 1:10 to 1:1, 1:5 to 1:1, and 40% to 60% mixture by weight loaded into the oil.

In a preferred embodiment, the formulation is a mixture of solid Turmeric extract and solid Boswellia extract, and the mixture is loaded into an oil. The Turmeric extract is enriched with curcuminoids with at least 80% curcuminoids and up to 98% curcuminoids w/w. Curcuminoids consist of curcumin, bisdemethoxycurcumin, and demethoxycurcumin. The solid Boswellia extract with enriched Boswellia acids and at least 10% of the extract is AKBA w/w. More preferably the formulation is made of the Turmeric extract—10 to 50%, the Boswellia extract—1 to 30%, and a Sesame oil—40 to 90% by weight. More preferably the formulation is made of the Turmeric extract about—30±2%, the Boswellia extract about-10±2%, and the Sesame oil about—60±2%.

In a preferred embodiment, the formulation also includes natural preservatives, which are selected from a group consisting of Cinnamon Bark Oil, Citric Acid, rosemary extract, Clove bud oil, celery seed oil, Oregano, Cardamom, Vanilla, Nutmeg fruit extract, Cumin seed, Ascorbic acid, Cinnamon leaf, and Cassia Extract. Said natural preservatives makes 0.1 to 10% of the formulation by weight or more precisely the preservatives in the formulation could be in 100-5000 ppm.

In yet another embodiment, curcuminoids and AKBA, or the solid Turmeric extract and solid Boswellia extract loaded in oil will have a particle size of about 100 nanometres to 200 microns. More than 90% of the particle dispersed in the oil will have a partial size ranging from 900 nanometres to 5 microns.

In one embodiment method of preparation of analgesic formulation is disclosed. The mixture is prepared by mixing solid Turmeric extract (about 95% curcuminoids) with solid Boswellia extract (about 10% AKBA and at least 30% Boswellic acid) in 2.8:1 ratio. The said mixture is added to an edible oil (sesame oil for this illustration) in 2.8:1:6.2 ratio and mixed well to get a high viscose blend. The blend thus made is passed through a bead mill. Anti-oxidants are dosed at 2000 ppm of the oil as preservatives which are a combination of rosemary and green tea extract. Then said blend is passed through the bead mill 5 times resulting an yellow coloured liquid with a viscosity in the range of 270 to 320 mP·s.

### The Activity of the Formulation

Another aspect of the invention is the method of hastening the onset of analgesia in a subject experiencing acute musculoskeletal pain. The subject is administered an effective dose of a mixture of solid Turmeric extract and solid Boswellia extract, the mixture is loaded in oil with a Hydrophilic-lipophilic balance value ranging from 10 to 19. The solid Turmeric extract and solid Boswellia extract is in 1:1 to 3:1 w/w ratio in the formulation, the preferred combination of solid Turmeric extract and solid Boswellia extract for an analgesic activity is 2.8:1 to 3:1. “Loading” or “loaded” refer to uniform mixing of solid compounds into an immiscible liquid, this could be achieved by physical force applied to the mixture to form a uniform dispersant. The subject is a human or an animal, for a human the onset of analgesia is reached within 30 to 210 minutes when administered with an effective dose of a mixture of solid Turmeric extract and solid Boswellia extract, and the mixture is loaded in an oil. The oil to be used is an edible oil selected from a group consisting but not limited to sesame oil, coconut oil, palm oil, olive oil, sunflower oil, mustard oil, lemongrass oil, bran oil, groundnut oil, lavender oil, flaxseed oil, grape seed oil, almond oil, avocado oil, soyabean oil, walnut oil, cashew oil, canola oil, corn oil, safflower oil, cottonseed oil, ramtil oil, niger oil, hemp oil, Turmeric oil, ginger oil, animal fat, fish oil, medium-chain triglycerides, saturated fatty acids, unsaturated fatty acid, or combination thereof. The mixture to the oil is in 1:99 to 3:2 w/w ratio, the mixture is uniformly dispersed in oil; a more preferred blend of the mixture to oil will be 1:10 to 1:1, 1:5 to 1:1, and 40% to 60% mixture by weight loaded into the oil.

In an embodiment, the method of hastening the onset of analgesia in a subject experiencing acute musculoskeletal pain includes administering a formulation of solid Turmeric extract and solid Boswellia extract at a dose of 200 mg to 800 mg. The said formulation is a mixture of solid Turmeric extract and solid Boswellia extract in 1:1 to 3:1 w/w ratio, and the mixture is loaded into an edible oil; ideally, the mean partial size of the loaded mixture in the oil is in the range of 100 nanometres to 200 microns.

In an embodiment, the subject is a human or an animal, for a human the onset of analgesia is reached within 30 to 210 minutes of oral administration of the formulation.

In an embodiment, all the formulations disclosed above are made into an oral dosage form, the dosage form is selected from a group of soft gel capsule, hard shell capsule, tablet, mini-tablet, granule, sachet, powder, paste, infusion, ampoule, solution, suspension, emulsion, pills or cream. The dosage form may be packed in 500 to 1000 mg dosage units, with each unit having about 200 mg to 800 mg of the mixture and the rest edible oil.

The treatment group showed a significant reduction in pain intensity (SPID6rest p<0.001) with a 97.85% improvement in cumulative responder analysis (AUC method) compared to 2.46% in placebo. The onset of pain relief is fast and highly significant in the treatment group with 99% of subjects having a mean PPR at 68.5 min and 96% of subjects having mean MPR at 191.6 min compared to the placebo group. Highly significant and continuous improvement in pain relief was observed in the treatment group with 93% of subjects having ≥50% of maximum TOTPAR6 with a number needed to treat of 1.1 at rest. The treatment group had clinically significant improvement in standardized response mean for SF-MPQ scores of total 2.31, sensory 1.77 and affective 1.15; Visual analogy score 5.51; and present pain index 4.88 compared to placebo.

Musculoskeletal Pain could be exercise-induced and is selected from a group comprising of low back pain, general whole-body pain, myalgia, headache, neck pain, limb pain, grade one sprain, joint pain, acute soft tissue injury, acute injuries of ligaments and acute injuries of tendons. The fast and sustained pain relief could be the result of blocking multiple pain pathways at once. Synergy was observed between curcuminoids and Boswellia acids compared to their actions.

In an embodiment, the method of hastening the onset of analgesia in a subject experiencing acute musculoskeletal pain in different body parts includes administering a formulation of Turmeric and Boswellia at a dose of 1000 mg. In all the five locations of pain and three modalities of rest, movement and pressure, head and neck, upper and lower limbs, and trunk, treatment group showed a significant pain intensity decrease compared to placebo. Treatment group shows a 100% relief in pain intensity in head and neck using Numerical Rating Scale pain after 6 hrs. The pain relief started as early as 40 minutes and complete pain relief was achieved as early as 160 min. In upper limb and trunk category, treatment group shows a 99% and 97% relief in pain intensity after 6 hrs. Upper limb and trunk category has a close PPR at 52 and 76 minutes and MPR is 168 and 216 minutes respectively. In the general pain category, treatment group had a perceptible pain relief as early as 75 minutes and a complete pain relief by 240 minutes. Lower limb category is close by 74 min in PPR and 176 min in MPR and 97% relief in pain intensity is shown after 6 hrs.

In another preferred embodiment, fast pain relief in subjects with acute musculoskeletal pain is achieved by administering pain relief formulation. There is a significant difference in LSM PID of treatment from 270 min postdose onwards. Sum of pain intensity difference (SPID6) and the area under the pain intensity effect curve (AUE6) showed a significant difference at 360 min when compared to placebo. In the cumulative responder analysis 61.93% of improvement is observed in the treatment group while it is 2.7% improvement in placebo. The mean TOTPAR at 6 h is 198 for the treatment group and 32 for the placebo. There is a statistically significant difference in the pain relief between the treatment group and placebo at 360 min.

The Restricted Mean Survival Time (RMST) of treatment group is 269 minutes compared to 312 minutes in placebo and pain relief is significantly better than placebo. The quality of pain assessed by McGill questionnaire is analysed using the Wilcoxon and paired t test. There is a significant difference in the treatment group in the sensory, affective domains and total score of MPQ whereas the placebo group has no significant change. In the VAS and PPI also the treatment group has significant difference whereas the placebo is without significant change.

In one embodiment efficacy of fast pain relief formulation in menstrual Cramp pain associated with primary Dysmenorrhea is disclosed. Sum of pain intensity difference (SPID6) showed a significant difference at 360 min when compared to placebo. The mean TOTPAR at 6 hours is 19 for the treatment group and 2 for the placebo. There is a statistically significant difference in pain relief between the treatment group and placebo at 360 min. In the treatment group, 73.3% reported that the formulation is an excellent pain reliever and 26.7% reported the formulation is very good for pain relief. Whereas in the placebo group 83.3% reported poor pain relief.

In another embodiment efficacy of the formulation of Turmeric and Boswellia extracts in sesame oil was compared to Turmeric and Boswellia extract powder for exercise-induced acute musculoskeletal pain. Dose administered was single dose of 2*500 mg capsule. The sum of pain intensity differences at 6 h showed that treatment group had a significant decrease in pain intensity at rest, pressure and movement (SPID6rest, SPID6pres, SPID6mov) compared to extract powder and Placebo. The mean total pain relief after 6 hours in treatment group was significantly better than the extract powder group. At rest, pressure and movement 98% subjects reported a pain relief of greater than 50% of Max TOTPAR in the treatment group, whereas only 6% reported that in Placebo. The extract powder group had 6% in rest and pressure but only 2% reported greater than 50% Max TOTPAR on movement.

### EXAMPLES

Specific embodiments of the invention will now be demonstrated by reference to the following general methods of manufacture and examples. It should be understood that these examples are disclosed solely by way of illustrating the invention and should not be taken in any way to limit the scope of the present invention. They are in no way limiting on the preceding description.

### Example 1

The Process to Manufacture the Formulation with a 3:1 Ratio of Turmeric to Boswellia Extract

The mixture was prepared by mixing 28 Kg of a solid Turmeric extract (about 95% curcuminoids) with 10 Kg of a solid Boswellia extract (about 10% AKBA and at least 30% Boswellic acid). The said mixture was added to a 62 Kg edible oil (sesame oil for this illustration) and mixed well to get a high viscose blend. The blend thus made was passed through a bead mill, in the bead mill the strong inter-movements of grinding media form great shearing, pressing and abrasive force, and the material was pulverized. As preservative anti-oxidants were dosed at 2000 ppm of the oil, preservatives were a combination of rosemary and green tea extract. The blend was passed through the bead mill 5 times and a yellow coloured liquid with a viscosity in the range of 270 to 320 mP·s was obtained (it shall be called sample 1).

### Example 2

The Process to Manufacture the Formulation with 10:1 Ratio of Curcuminoids to Boswellic Acid

The mixture was prepared by mixing 10 Kg of a solid curcuminoid (about 98% purity) with 1 Kg of a solid Boswellia acid (about 34% AKBA). The said mixture was added to a 17 Kg edible oil (sesame oil for this illustration) and mixed well to get a high viscose blend. The blend thus made was passed through a bead mill, in the bead mill the strong inter-movements of grinding media form great shearing, pressing and abrasive force, and the material was pulverized. As preservative anti-oxidants were dosed at 2000 ppm of the oil, preservatives were a combination of rosemary and green tea extract. The blend was passed through the bead mill 5 times to get the final formulation (Sample 1), the formulation was yellow coloured liquid with a viscosity in the range of 270 to 320 mP·s.

### Example 3

Sample 1 from example 1 was made into a dosage form by filling it into a soft gel capsule. The soft gel capsule had a unit dose of 500 mg of sample 1. Each capsule contained about 133 mg curcuminoids and 5 mg AKBA.

Composition of 500 mg soft gel capsule

| | | |
|---|---|---|
| Turmeric extract | 140 | mg (Curcuminoids 133 mg) |
| Boswellia extract | 50 | mg (AKBA 5 mg) |
| Sesame oil | 310 | mg |
| Total | 500 | mg |

### Example 4

### Fast Pain Relief in the Event of Exercise-Induced Acute Musculoskeletal Pain (MSP) by Administering Pain Relief Formulation

This multicentric study analysed the efficacy of a formulation of Turmeric and Boswellia extracts in sesame oil made as per example 1. For this study 232 subjects were randomized in an allocation concealed 1:1 ratio to receive a single dose of 1000 mg of Sample 1 (treatment) or placebo. The study included healthy male and female subjects aged 18-65 years with exercise-induced acute MSP with a resting NRS of 5 or above on a 0-10 scale, which occurred within 24 hours before presenting at the site. The subjects who met all the inclusion and exclusion criteria were randomized to receive either the test intervention Sample 1, 1000 mg (500 mg×2 soft-gels) containing 266 mg curcuminoids and 10 mg acetyl keto-Boswellia acids or comparator intervention of matching Placebo 1000 mg. The investigators and the subjects were blinded using a placebo with similar size, colour, packaging, and labelling. All staff engaged with the study were blinded to the identity of the treatments.

The primary outcome was a change in the Sum of Pain Intensity Difference at 6 hours at rest (SPID6ᵣₑₛₜ) calculated from the numerical rating scale (NRS). The secondary outcomes were the time to perceptible pain relief (PPR) and time to meaningful pain relief (MPR) using double-stopwatch method (onset to analgesia); SPID6 at movement (SPID6ₘₒᵥₑ) and pressure (SPID6ₚᵣₑₛ); total pain relief at 6 h at rest (TOTPAR6ᵣₑₛₜ), movement (TOTPAR6ₘₒᵥₑ) and pressure (TOTPAR6ₚᵣₑₛ) using pain relief scale (PRS) and the change in the quality of pain using a short form of McGill Short Form Questionnaire (SF-MPQ). The outcomes were assessed without unblinding the interventions. The derived outcomes from NRS scores were pain intensity difference (PID), SPID at 180 and 360 minutes; Area under the PID curve at 180 and 360 minutes and cumulative proportion of responder's analysis (CPRA) from the AUC curve for rest, movement, and pressure. The number needed to treat (NNT) was calculated as an additional outcome of PRS_{rest, move, pres}.

### Numerical Pain Rating Scale (NRS)

The NRS is an 11-point scale in which 0 represents ‘no pain’ and 10 represents the worst pain possible. The rating score of 1-4 is considered as mild, 5-6 is moderate and 7-10 is severe pain The NRS was taken at 30 min intervals up to 6 hours at rest (NRSrest), on the movement of the affected part (NRSmove), and on applying pressure to the affected part for calculating the SPID6_{rest,move,pres}.

### Pain Relief Scale (PRS)

The pain relief scale is a categorical scale having a positive progression from ‘No relief’, ‘A little relief’, ‘Some relief’, ‘A lot of relief’ to ‘Complete relief’ (coded 0 to 4). TOTPAR is the area under the time-analgesic effect curve for a given time. The PRS was taken at 30 min intervals up to 6 hours for calculating the TOTPAR6. The PRS was taken when the subjects were at rest (PRSᵣₑₛₜ), on the movement of the affected part (PRSmove), and on applying pressure to the affected part (PRSₚᵣₑₛ).

### Onset to Analgesia

The onset to analgesia was taken using the “double stopwatch” method. After dosing, two stopwatches were started simultaneously. The first stopwatch was stopped when the subject reported the first perception of pain relief (PPR). The second stopwatch was stopped when the subject felt complete pain relief called meaningful pain relief (MPR). The time to PPR and MPR was recorded in hours and minutes and when PPR/MPR was not reached within 6 h, it was censored at that time point.

### Short Form of McGill Pain Questionnaire (SF-MPQ)

The McGill Pain Questionnaire (MPQ) allows the subject to describe the quality (affective domain and intensity (Sensory domain) of pain and were answered at baseline and at the end of the study. Participants also rated their present pain intensity (PPI) on a 0 to 5 scale. Pain intensity was assessed on a 0 to 100 mm horizontal VAS, anchored by no pain (score of 0) and worst possible pain (score of 100).

In the study, 235 subjects were screened, and 232 subjects were enrolled with four sites enrolling 38 each, and two sites enrolling 40 subjects each. There was no significant difference between the groups in the baseline parameters of NRSrest (p=0.053), NRSmove (p=0.357) and NRSpres (p=0.266). There was also no significant difference between the demographic variables of age (p=0.919), height (p=0.747) and weight (p=0.846). Demographics and baseline data of treatment and placebo groups are given in Table 1. The subjects enrolled had exercise-induced pain in lower back (n=70), shoulder (n=22), knee (n=20), lower body (n=22) musculoskeletal (n=6), and other types of pain (n=92). There were no adverse events and no dropouts in this study.

### NRS on Rest, Movement, and Pressure

Sum pain intensity differences (SPID) and area under the curve (AUC) of 180 and 360 minutes for NRS_{rest,move,pres}) showed a significant difference (p<0.001) between the treatment and placebo and is given in Table 1. In the AUC responder analysis using NRS_{rest,move,pres} registered a difference of 95.39%, 93.52% and 93.28% respectively more than placebo responders at the end of 6 h.

TABLE 1

| | | NRS Rest | NRS Movement | NRS Pressure |
|---|---|---|---|---|
| | | Mean | Mean | Mean |
| SPID | Treatment | 642 | 706 | 689 |
| (0-180) | Placebo | −15 | 17 | 24 |
| AUC | Treatment | 551 | 606 | 590 |
| (0-180) | Placebo | −14 | 16 | 22 |
| SPID | Treatment | 1944 | 2080 | 2072 |
| (0-360) | Placebo | −35 | 36 | 50 |
| AUC | Treatment | 1827 | 1956 | 1948 |
| (0-360) | Placebo | −33 | 35 | 48 |

The Least Square Mean (LSM) difference of treatment from placebo showed a statistically significant difference for all categories (NRS_{rest,move,pres}) from one hour onwards till the end of the study at the specified time points. The NRS analysis showed that treatment reduced acute pain intensity which was statistically and clinically significant. Responder Analysis and LSM PID of Treatment from Placebo for NRS_{rest,move,pres} is detailed in Table 2a, 2b and 2c.

TABLE 2a

| | NRS Rest | | | | | | |
|---|---|---|---|---|---|---|---|
| | Treatment | | | Placebo | | | |
| | | % | | | % | | LSM |
| Time | | Improve- | | | Improve- | | Difference |
| (h) | AUC | ment | LSM | AUC | ment | LSM | (P − T) |
| 1 | 2654 | 26.54 | 5.9 | 119 | 1.19 | 7.8 | 1.9 |
| 2 | 5776 | 57.76 | 3.7 | 115 | 1.15 | 7.8 | 4.1 |
| 3 | 7912 | 79.12 | 1.9 | 160 | 1.6 | 7.8 | 5.9 |
| 4 | 8766 | 87.66 | 1.1 | 190 | 1.9 | 7.8 | 6.7 |
| 5 | 9393 | 93.93 | 0.5 | 249 | 2.49 | 7.8 | 7.2 |
| 6 | 9785 | 97.85 | 0.2 | 246 | 2.46 | 7.8 | 7.6 |

TABLE 2b

| | NRS Movement | | | | | | |
|---|---|---|---|---|---|---|---|
| | Treatment | | | Placebo | | | |
| | | % | | | % | | LSM |
| Time | | Improve- | | | Improve- | | Difference |
| (h) | AUC | ment | LSM | AUC | ment | LSM | (P − T) |
| 1 | 2772 | 27.72 | 6.2 | 314 | 3.14 | 8.1 | 1.9 |
| 2 | 6007 | 60.07 | 3.6 | 315 | 3.15 | 8.2 | 4.6 |
| 3 | 8016 | 80.16 | 1.7 | 347 | 3.47 | 8.1 | 6.4 |
| 4 | 8791 | 87.91 | 1 | 366 | 3.66 | 8.1 | 7.1 |
| 5 | 9281 | 92.81 | 0.6 | 427 | 4.27 | 8.1 | 7.5 |
| 6 | 9779 | 97.79 | 0.2 | 427 | 4.27 | 8.2 | 8 |

TABLE 2c

| | NRS Pressure | | | | | | |
|---|---|---|---|---|---|---|---|
| | Treatment(T) | | | Placebo(P) | | | |
| | | % | | | % | | LSM |
| Time | | Improve- | | | Improve- | | Difference |
| (h) | AUC | ment | LSM | AUC | ment | LSM | (P − T) |
| 1 | 2589 | 25.89 | 6.2 | 351 | 3.51 | 8.1 | 1.9 |
| 2 | 5778 | 57.78 | 3.6 | 342 | 3.42 | 8.2 | 4.6 |
| 3 | 7944 | 79.44 | 1.7 | 381 | 3.81 | 8.1 | 6.4 |
| 4 | 8772 | 87.72 | 1 | 409 | 4.09 | 8.1 | 7.1 |
| 5 | 9397 | 93.97 | 0.6 | 456 | 4.56 | 8.1 | 7.5 |
| 6 | 9784 | 97.84 | 0.2 | 456 | 4.56 | 8.2 | 8 |

### Onset to Analgesia

The onset of analgesia was fast and highly significant in the treatment group with 99.1% of subjects experiencing a PPR and 95.7% subjects achieving MPR compared to 10.4% subjects experiencing PPR and 1.7% achieving MPR in the placebo group. As the pain relief was relatively low in the placebo group, the median pain relief time could not be estimated from the observed data. Restricted Mean Survival Time (RMST) analysis showed a PPR of 68.5 min and MPR of 191.6 min in the treatment group and 340.7 min and 358.1 min in the placebo group respectively. The RMST ratio indicates that a person in the treatment group experienced a PPR of 4.98 times faster and achieved an MPR of 1.87 times faster than a placebo. The RMLT ratio of 0.07 (PPR) and 0.01 (MPR) showed that the placebo group experienced 93% and 99% less pain-free time respectively. A low RMST or a high RMLT in MPR and PPR indicated a lesser duration of pain in the treatment group and showed an overall benefit compared to the placebo group.

From the Kaplan Meir (KM) survival plots for PPR, 45 (39%) subjects in the treatment group experienced PPR as early as 30 min and 115 (99%) in 190 min while in placebo 2 (1.7%) subjects experienced PPR as early as 30 min and 12 (10.3%) in 285 minutes. The earliest MPR was reached within 75 min (1 subject) and 111 (96%) subjects had MPR within 360 minutes while the placebo had only 2 events (1.7%) at 360 minutes.

The Log-rank hazard ratio for PPR and MPR indicated a significant difference in symptom resolution between the groups. Cox regression analysis estimated PPR and MPR with NRS rest as a covariate and showed a significant difference in symptom resolution indicating that a person receiving treatment was 83 and 272 times more likely to experience PPR and MPR compared to placebo whereas the corresponding figures for PPR and MPR in the log-rank test was 19 and 107 times. This strongly suggests that a difference between the groups exists which is clinically important.

### Pain Relief Scale (PRS)

TOTPAR6rest, move, the pres showed a significant difference between the two groups demonstrating better efficacy for treatment in obtaining pain relief. The effect of treatment in terms of NNT (a count of how many people need to be treated for one person to benefit) in the treatment group for 50% of maximum pain relief was 1.1 in rest, movement, and pressure. The number of subjects with ≥50% of Maximum TOTPAR was 108, 109 and 109 in the treatment group (p<0.001 for PRSrest, move, pres) compared to 3, 1 and 1 in placebo (Table 3).

TABLE 3
Comparison of Total pain relief between treatment
and placebo and number needed to treat

| | | | | Treatment | Placebo | |
|---|---|---|---|---|---|---|
| | | Treatment | Placebo | Proportion | Proportion | |
| | | (n) | (n) | (n/N) | (n/N) | NNT |
| PRSᵣₑₛₜ | ≥50% of Max | 108 | 3 | 0.93 | 0.02 | 1.10 |
| | <50% of Max TOTPAR | 8 | 113 | 0.06 | 0.97 | |
| PRSₘₒᵥₑ | ≥50% of Max | 109 | 1 | 0.93 | 0.01 | 1.07 |
| | <50% of Max TOTPAR | 7 | 115 | 0.06 | 0.99 | |
| PRSₚᵣₑₛ | ≥50% of Max | 109 | 1 | 0.93 | 0.01 | 1.07 |
| | <50% of Max TOTPAR | 7 | 115 | 0.06 | 0.99 | |

The RMST to achieve maximum pain relief by KM analysis at rest, movement and pressure was 194 min, 197.7 min, and 194.2 min for treatment compared to 345.5, 345.5, and 356.7 respectively for statistically significant placebo. A high RMST (Restricted Mean Survival Time to maximum pain relief) or low RMTL (Restricted Mean Time Lost to maximum pain relief) in PRS_{rest,move,pres} indicates faster pain relief in the treatment group and shows overall benefit compared with the placebo group (Table 4). In the treatment group, 50% of subjects experienced maximum pain relief of 180 min, 182 min, 180 min (median time) for rest, movement, and pressure respectively. The median time was not assessable in the placebo group.

TABLE 4
Restricted Mean Survival Time to achieve maximum Pain Relief and Restricted Mean
Time Lost for Pain-free time from the categorical pain relief scale (PRS).

| | | Subjects (n) | | | | |
|---|---|---|---|---|---|---|
| | | Symptom | | Time | RMST | RMTL |
| | Group | resolved | Total | (minutes) | (minutes) | (Minutes) |
| PRS on Rest | Treatment | 108 | 116 | 360 | 194 | 166 |
| | Placebo | 11 | 116 | 360 | 346 | 15 |
| PRS on Movement | Treatment | 114 | 116 | 360 | 198 | 162 |
| | Placebo | 9 | 116 | 360 | 353 | 7 |
| PRS on Pressure | Treatment | 110 | 116 | 363 | 194 | 169 |
| | Placebo | 8 | 116 | 363 | 357 | 7 |

The repeated measure proportional odds logistic regression analysis showed an increase in pain relief with time represented by a negative time coefficient which was significant in both treatments and placebo. A significant effect can be seen in the placebo group with this analysis but the odds of experiencing complete pain relief in the same period is negligible compared to the treatment. The cumulative probability odds of getting complete pain relief with placebo compared to treatment was 0.00025, 0.00025 and 0.00024 times for rest, movement, and pressure respectively (Table 5).

TABLE 5
Numbers of subjects according to the category of pain relief

| | | Numbers of subjects | | | | |
|---|---|---|---|---|---|---|
| | | None | Little | Some | A Lot | Complete |
| PRS at | Treatment | 1 | 0 | 0 | 7 | 108 |
| Rest | Placebo | 105 | 10 | 0 | 0 | 1 |
| PRS at | Treatment | 1 | 0 | 0 | 9 | 106 |
| Movement | Placebo | 107 | 8 | 0 | 0 | 1 |
| PRS at | Treatment | 1 | 0 | 0 | 7 | 108 |
| Pressure | Placebo | 108 | 7 | 0 | 0 | 1 |

### Short Form of McGill Pain Questionnaire (SF-MPQ)

The treatment group showed a statistically significant reduction in total, sensory and affective domains of McGill Pain Questionnaire (MPQ), Visual Analog Scale (VAS), present pain index (PPI) comparing post-treatment with pre-treatment while placebo had no significant change in MPQ (p>0.05) whereas a significant change was observed in VAS and PPI. SRM value greater than 0.80 shows a clinically significant improvement and the treatment group had a value greater than 0.8 in MPQ, VAS and PPI while the placebo group had less than 0.5 in MPQ, VAS and PPI indicating no beneficial change. A change of 13.7 mm in VAS shows a minimum clinically important difference (MCID) and the treatment group showed a reduction greater than MCID (mean difference 78.6 mm) whereas the change in placebo was less than MCID (Table 6).

TABLE 6
McGill short-form questionnaire comparison
between treatment and placebo group.

| | | Treatment | Placebo |
|---|---|---|---|
| | | Mean | Mean |
| MPQ Sensory | Pre-treatment | 11 | 11 |
| (Scale 0-33) | Post Treatment | 0.2 | 11 |
| MPQ Affective | Pre-treatment | 3 | 3 |
| (Scale 0-12) | Post Treatment | 0.1 | 3 |
| Total MPQ (Scale | Pre-treatment | 14 | 14 |
| 0-45) | Post Treatment | 0.3 | 14 |
| VAS | Pre-treatment | 81 | 81 |
| | Post Treatment | 2 | 82 |
| PPI | Pre-treatment | 4 | 4 |
| | Post Treatment | 0.2 | 4 |

Most of the scales used in the assessment of acute pain management are unidimensional like NRS and VAS and measure pain intensity. The pain being multidimensional using either of them alone in a study does not give the whole picture. PRS taken together with NRS for the evaluation of pain increased the objectivity of pain assessment better than using only VAS or NRS. The objectivity of the assessment was further increased by determining the time to onset of analgesia using the double-stopwatch method. To ascertain the multidimensional nature of pain the short form of the McGill pain questionnaire was used. The change in NRS pain score and its effect on pain relief in patients with moderate pain showed that a reduction of 1.3, 2.4, and 3.5 units in NRS corresponded to minimal, much, and very much improvement in the degree of pain relief.

Most of the clinical trials using NRS tend to consider only the pain relief at rest or movement separately which severely underestimates the reduction in pain intensity and leads to reporting bias. So, in this study pain intensity reduction was measured not only at rest but also on movement of the affected part and on the pressure applied to determine the overall efficacy of the treatment. It can be seen that majority of subjects had more NRS scores for movement and pressure than for rest at baseline. The primary efficacy variable NRS at rest showed a significant reduction in pain intensity (SPID 180 and 360) with 95% responders compared to placebo. The fact that movement had almost similar pain relief indicates that the treatment was effective in improving the function of the affected part. The improvement in pain intensity reduction when pressure was applied to the affected part indicated that the treatment was effective in mitigating pain due to acute inflammation.

### Example 5

### Fast Pain Relief in Exercise-Induced Acute Musculoskeletal Pain in Different Body Parts by Administering Pain Relief Formulation

This randomized, placebo-controlled, multicentre study was conducted for analysing the efficacy of a formulation of Turmeric and Boswellia extracts in sesame oil made as per example 1 for acute musculoskeletal pain in different parts of the body viz., head and neck, trunk, upper and lower limbs. For this study 232 healthy subjects of both male and female aged 18-65 years with acute musculoskeletal pain which occurred within 24 hours before presenting at the site were randomized in 1:1 ratio to receive a single dose of 1000 mg of Sample 1 (treatment) or placebo. The participants were categorized according to the location of the pain-head and neck, upper limb, lower limb, trunk, and general body following exercise. The intensity of pain was measured using a numerical rating scale (NRS) and subjects with greater than or equal to 5 are considered eligible for enrolment. The subjects who met all the inclusion and exclusion criteria were randomized to receive either the test intervention Sample 1, 1000 mg (500 mg×2 soft-gels) containing 266 mg curcuminoids and 10 mg acetyl keto-Boswellia acids or comparator intervention of matching Placebo 1000 mg. The investigators and the subjects were blinded using a placebo with similar size, colour, packaging, and labelling. All staff engaged with the study were blinded to the identity of the treatments.

The study focused on analysing the data segregated based on the location of musculoskeletal pain reported by the subjects upon entry into the study. The location of pain was segregated into 5 categories.
Head and neck: Neck
Trunk: Back, Pelvic
Upper limb: Shoulder, Hand, Arm, Forearm, Clavicle, Wrist, Elbow
Lower limb: Hip and Thigh Muscles, Leg, Foot Muscles, Knees, Ankle
General musculoskeletal pain

The primary objective of the study was to determine the efficacy of the formulation by measuring the pain intensity. The pain intensity was measured by a numerical pain rating scale (NRS). The NRS is an 11-point scale in which 0 represents ‘no pain’ and 10 represents the worst pain possible. The rating score of 1-4 is considered as mild, 5-6 is moderate and 7-10 is severe pain. The NRS was taken post dose every 30 minutes up to 6 hours when the subject was seated at rest (NRSrest), on the movement of the affected part (NRSmove), and on applying pressure to the affected part. The sum of pain intensity difference was calculated for 6 hours (SPID6) from baseline.

The onset to analgesia was taken using the “double stopwatch” method. After dosing, two stopwatches were started simultaneously. The first stopwatch was stopped when the subject reported the first perception of pain relief (PPR). The second stopwatch was stopped when the subject felt complete pain relief called meaningful pain relief (MPR). Median Survival Time and Restricted Mean Survival Time (RMST) analysis of onset to analgesia was done. The restricted mean survival time (symptom resolution time) is a measure of treatment effect wherein the average time of a subject who continues in pain from time 0 to cessation of pain at time t is taken. The RMST difference measures the effect of treatment on the restricted symptom resolution time at t. The RMST value can also be an absolute measure of symptom resolution time, this dual mode of presentation as both an absolute and a relative measure is an added advantage of this measure.

Total 232 subjects were enrolled and subjects categorized into 5 groups based on the location of pain and most common location of musculoskeletal pain was trunk (n=85) and lower limb (n=76) followed by upper limb (n=47). 14 subjects have head and neck pain and 10 subjects has general musculoskeletal pain. The location-wise distribution of pain was statistically not different in both treatment and placebo groups. There was no significant difference in the distribution of pain gender-wise and location-wise in both groups.

The pain intensity decreased in the head and neck, lower limb, trunk, upper limb, and general pain category were highly significant in the treatment group in the three modalities of rest, movement and pressure. In the generalized musculoskeletal pain (n=4) there was a near significant decrease in all the three modalities of NRS measurement.

Treatment response over 360 minutes (6 hrs) was summarised as a sum of pain intensity difference (SPID6). In the study, the treatment response of treatment group was significantly better than the placebo at rest, on movement, and on pressure. In all the five categories of general musculoskeletal pain, head and neck, lower and upper limbs, and trunk, treatment group showed a significant pain intensity decrease compared to placebo. In the placebo group there was no significant decrease in any of the 5 categories of pain location.

### Head and Neck

TABLE 7
Analysis of Pain Intensity in head and neck Score
using Numerical Rating Scale in treatment group

| | Treatment | | | Placebo | | |
|---|---|---|---|---|---|---|
| | | End | % | | End | % |
| NRS | Baseline | (6 Hrs) | change | Baseline | (6 Hrs) | change |
| rest | 7.50 | 0 | 100 | 7.63 | 7.75 | −1.64 |
| move | 7.83 | 0 | 100 | 8.50 | 7.75 | +8.82 |
| pressure | 7.83 | 0 | 100 | 8.25 | 7.75 | +6.06 |

In treatment group, a 100% relief in pain intensity in head and neck using Numerical Rating Scale pain after 6 hrs.

TABLE 8
Analysis of Sum of Pain Intensity Difference at 6 hours (SPID6)
between treatment and Placebo groups

| SPID6 | Placebo | Treatment | Mean difference |
|---|---|---|---|
| rest | −56 | 2220 | −2276 |
| move | 241 | 2305 | −2064 |
| pressure | 154 | 2298 | −2143 |

TABLE 9
Median Survival Time, Restricted Mean Survival Time analysis
of onset to analgesia using the double-stopwatch method

| | Perceptible Pain Relief | | Meaningful Pain Relief | |
|---|---|---|---|---|
| | (PPR) | | (MPR) | |
| Parameter | Treatment | Placebo | Treatment | Placebo |
| Symptom resolved | 6 of 6 | 2 of 8 | 6 of 6 | 0 of 8 |
| (n of N) | | | | |
| Median Survival | 30 | NE | 120 | NE |
| Time | | | | |
| Restricted Mean | 40 | 60 | 160 | 360 |
| Survival Time | | | | |
| (RMST) | | | | |

| | | |
|---|---|---|
| RMST Difference | 20 | 200 |
| (P − T) | | |
| RMST Ratio (P/T) | 1.5 | 2.25 |

Subjects in the head and neck category had the fastest PPR at 40 minutes and an MPR of 160 min.

### Upper Limb

TABLE 10
Analysis of Pain Intensity in upper limb Score
using Numerical Rating Scale in treatment group

| | Treatment | | | Placebo | | |
|---|---|---|---|---|---|---|
| | | End | % | | End | % |
| NRS | Baseline | (6 Hrs) | change | Baseline | (6 Hrs) | change |
| rest | 7.46 | 0.08 | 98.88 | 7.30 | 7.43 | −1.79 |
| move | 8.25 | 0.08 | 98.99 | 7.87 | 7.96 | −1.10 |
| pressure | 8.33 | 0.08 | 99 | 8.09 | 8.00 | 1.08 |

TABLE 11
Analysis of Sum of Pain Intensity Difference at 6 hours (SPID6)
between treatment and Placebo groups

| SPID6 | Placebo | Treatment | Mean difference |
|---|---|---|---|
| rest | −56 | 2030 | −2086 |
| move | −46 | 2244 | −2290 |
| pressure | 16 | 2253 | −2237 |

TABLE 12
Median Survival Time, Restricted Mean Survival Time analysis
of onset to analgesia using the double-stopwatch method

| | Perceptible Pain Relief | | Meaningful Pain Relief | |
|---|---|---|---|---|
| Parameter | Treatment | Placebo | Treatment | Placebo |
| Symptom resolved | 24 of 24 | 2 of 23 | 23 of 24 | 0 of 23 |
| (n of N) | | | | |
| Median Survival | 30 | NE | 150 | NE |
| Time | | | | |
| Restricted Mean | 52 | 143 | 168 | 360 |
| Survival Time | | | | |
| (RMST) | | | | |

| | | |
|---|---|---|
| RMST Difference | 91 | 192 |
| (P − T) (p-value) | | |
| RMST Ratio (P/T) | 2.75 | 2.14 |

The upper limb category had a close PPR at 52 and the MPR was 168 respectively.

### Trunk

TABLE 13
Analysis of Pain Intensity in trunk Score using
Numerical Rating Scale in treatment group

| | treatment | | | Placebo | | |
|---|---|---|---|---|---|---|
| | | End | % | | End | % |
| NRS | Baseline | (6 Hrs) | change | Baseline | (6 Hrs) | change |
| rest | 8.30 | 0.26 | 97 | 7.87 | 7.95 | −0.98 |
| move | 8.61 | 0.24 | 97 | 8.44 | 8.31 | 1.52 |
| pressure | 8.59 | 0.26 | 97 | 8.41 | 8.21 | 2.44 |

TABLE 14
Analysis of Sum of Pain Intensity Difference at 6 hours (SPID6)
between treatment and Placebo groups

| SPID6 | Placebo | Treatment | Mean difference |
|---|---|---|---|
| rest | −29 | 1803 | −1832 |
| move | 48 | 1918 | −1870 |
| pressure | 69 | 1898 | −1829 |

TABLE 15
Median Survival Time, Restricted Mean Survival Time analysis
of onset to analgesia using the double-stopwatch method

| | Perceptible Pain Relief | | Meaningful Pain Relief | |
|---|---|---|---|---|
| Parameter | Treatment | Placebo | Treatment | Placebo |
| Symptom | 46 of 46 | 1 of 39 | 43 of 46 | 0 of 39 |
| resolved (n of N) | | | | |
| Median Survival | 60 | NE | 200 | NE |
| Time | | | | |
| Restricted Mean | 76 | 190 | 216 | 360 |
| Survival Time | | | | |
| (RMST) | | | | |

| | | |
|---|---|---|
| RMST Difference | 114 | 144 |
| (P − T) | | |
| RMST Ratio | 2.5 | 1.7 |
| (P/T) | | |

The trunk category had a close PPR at 76 and the MPR was 216 min respectively.

### Lower Limb

TABLE 16
Analysis of Pain Intensity in lower limb Score
using Numerical Rating Scale in treatment group

| | treatment | | | Placebo | | |
|---|---|---|---|---|---|---|
| | | End | % | | End | % |
| NRS | Baseline | (6 Hrs) | Change | Baseline | (6 Hrs) | Change |
| rest | 7.92 | 0.22 | 97 | 7.83 | 7.83 | 0 |
| move | 8.31 | 0.28 | 97 | 8.43 | 8.25 | 2.08 |
| pressure | 8.39 | 0.22 | 97 | 8.38 | 8.20 | 2.09 |

TABLE 17
Analysis of Sum of Pain Intensity Difference at 6 hours (SPID6)
between treatment and Placebo groups

| SPID6 | Placebo | Treatment | Mean difference |
|---|---|---|---|
| rest | 3 | 2010 | −2007 |
| move | 50 | 2109 | −2059 |
| pressure | 52 | 2134 | −2082 |

TABLE 18
Median Survival Time, Restricted Mean Survival Time analysis
of onset to analgesia using the double-stopwatch method

| | Perceptible Pain Relief | | Meaningful Pain Relief | |
|---|---|---|---|---|
| Parameter | Treatment | Placebo | Treatment | Placebo |
| Symptom | 35 of 36 | 6 of 40 | 35 of 36 | 1 of 40 |
| resolved (n of N) | | | | |
| Median Survival | 60 | NE | 150 | NE |
| Time | | | | |
| Restricted Mean | 74 | 329 | 176 | 355 |
| Survival Time | | | | |
| (RMST) | | | | |

| | | |
|---|---|---|
| RMST | 255 | 179 |
| Difference (P − T) | | |
| RMST Ratio | 4.4 | 2 |
| (P/T) | | |

Lower limb category was close by 74 min in PPR and 176 min in MPR.

In the placebo group there was no significant decrease in any of the 5 categories of pain location.

### General Pain

TABLE 19
Analysis of Pain Intensity in general Score using
Numerical Rating Scale in the treatment group

| | Treatment | | | Placebo | | |
|---|---|---|---|---|---|---|
| | | End | % | | End | % |
| NRS | Baseline | (6 Hrs) | change | Baseline | (6 Hrs) | change |
| rest | 8.3 | 0 | 100 | 6.7 | 7.7 | −15 |
| move | 9 | 0 | 100 | 7.7 | 8.2 | −6.5 |
| pressure | 8.8 | 0.25 | 97 | 7.8 | 8.3 | −6.4 |

TABLE 20
Analysis of Sum of Pain Intensity Difference at 6 hours (SPID6)
between treatment and Placebo groups

| | | | Mean |
|---|---|---|---|
| SPID6 | Placebo | Treatment | difference |
| rest | −210 | 2040 | −2250 |
| move | −90 | 2358 | −2448 |
| pressure | −90 | 2100 | −2190 |

TABLE 21
Median Survival Time, Restricted Mean Survival Time analysis
of onset to analgesia using the double-stopwatch method

| | Perceptible Pain Relief | | Meaningful Pain Relief | |
|---|---|---|---|---|
| Parameter | Treatment | Placebo | Treatment | Placebo |
| Symptom | 4 of 4 | 1 of 6 | 4 of 4 | 1 of 6 |
| resolved (n of N) | | | | |
| Median Survival | 60 | NE | 180 | NE |
| Time | | | | |
| Restricted Mean | 75 | 140 | 240 | 358 |
| Survival Time | | | | |
| (RMST ± SE) | | | | |

| | | |
|---|---|---|
| RMST Difference | 65 | 118 |
| (P − T) | | |
| RMST Ratio | 1.87 | 1.49 |
| (P/T) ± SE | | |

In all the five locations of pain and three modalities of rest, movement and pressure, head and neck, upper and lower limbs, and trunk, treatment group showed a significant pain intensity decrease compared to placebo. Test formulation is safe and effective for acute pain irrespective of the location of the pain. In conclusion, treatment group was effective in reducing the pain at locations of head and neck, upper limb, lower limb, trunk and general body. In the five major locations of pain, the treatment group had above 96% reduction in pain intensity in all the modalities of rest, movement, and pressure whereas the placebo group showed negligent change. The study findings suggest that the efficacy of test formulation may be actively considered as a fast relief for acute pain irrespective of the location of the pain. Importantly, formulation could potentially be a safe alternative to analgesics commonly recommended for acute pain such as NSAIDs.

### Example 6

Fast Pain Relief in Subjects with Acute Musculoskeletal Pain by Administering Pain Relief Formulation

This randomized, placebo-controlled, study was conducted for analysing the efficacy of a formulation of Turmeric and Boswellia extracts in sesame oil for acute musculoskeletal pain. For this study, 40 healthy male and female subjects aged 18-65 years with acute musculoskeletal pain which occurred within 24 hours before presenting at the site were randomized in 1:1 ratio to receive a single dose 500 mg of test formulation or placebo. The primary outcome was the sum of pain intensity difference at 6 hours (SPID6) evaluated by Numerical Rating Scale (NRS) and total pain relief (TOTPAR6) by categorical pain relief scale (PRS) every 30 minutes up to 6 hours. The secondary outcomes were perceptible and meaningful pain relief (PPR and MPR) and sensory and affective pain using McGill Pain Questionnaire-Short Form (MPQ-SF).

The NRS is an 11-point scale in which 0 represents ‘no pain’ and 10 represents the worst pain possible. The rating score of 1-4 is considered as mild, 5-6 is moderate and 7-10 is severe pain. The NRS was taken at 30 min intervals up to 6 hours for calculating the SPID6. The pain relief scale (PRS) is a categorical scale having a positive progression from ‘No relief’, ‘A little relief’, ‘Some relief’, ‘A lot of relief’ to ‘Complete relief’ (coded 0 to 4). TOTPAR is the area under the time-analgesic effect curve for a given time. The PRS was taken at 30 min intervals up to 6 hours for calculating the TOTPAR6. The onset to analgesia was taken using the “double stopwatch” method. After dosing, two stopwatches were started simultaneously. The first stopwatch was stopped when the subject reported the first perception of pain relief (PPR). The second stopwatch was stopped when the subject felt complete pain relief called meaningful pain relief (MPR).

The McGill Pain Questionnaire (MPQ) allows the subject to describe the quality (affective domain and intensity (Sensory domain) of pain and were answered at baseline and at the end of the study. Participants also rated their present pain intensity (PPI) on a 0 to 5 scale. Pain intensity was assessed on a 0 to 100 mm horizontal VAS, anchored by no pain (score of 0) and worst possible pain (score of 100).

TABLE 22
Pain intensity assessed using Numerical rating scale

| Test formulation | | | Placebo | | |
|---|---|---|---|---|---|
| Time(min) | Mean | LSM PID | Time (min) | Mean NRS | LSM PID |
| Pre Dose | 6 | 0 | Pre Dose | 6 | 0 |
| 30 | 6 | 0 | 30 | 6 | 0 |
| 60 | 6 | 0 | 60 | 6 | 0 |
| 90 | 6 | 0 | 90 | 6 | 0 |
| 120 | 6 | 0 | 120 | 6 | 0 |
| 150 | 6 | 0 | 150 | 6 | 0 |
| 180 | 6 | 0.1 | 180 | 6 | 0.05 |
| 210 | 6 | 0.2 | 210 | 6 | 0.1 |
| 240 | 6 | 0.4 | 240 | 6 | 0.15 |
| 270 | 6 | 0.5 | 270 | 6 | 0.15 |
| 300 | 5 | 1.3 | 300 | 6 | 0.15 |
| 330 | 4 | 2.45 | 330 | 6 | 0.15 |
| 360 | 2 | 3.75 | 360 | 6 | 0.15 |

There is a significant difference in LSM of treatment from 270 min postdose onwards.

Sum of pain intensity difference (SPID6) and the area under the pain intensity effect curve (AUE6) showed a significant difference at 360 min when compared to placebo.

TABLE 23
Comparison of sum of pain intensity difference (SPID)
at 180 min and 360 minutes for the two groups.

| | SPID 180 | | SPID 360 | | AUE 180 | | AUE 360 | |
|---|---|---|---|---|---|---|---|---|
| | Treatment | Placebo | Treatment | Placebo | Treatment | Placebo | Treatment | Placebo |
| Mean SPID | 3 | 1.5 | 261 | 27 | 1.5 | 0.75 | 205 | 25 |
| LSM Diff | −1.5 | | −234 | | −0.75 | | −180 | |
| from Placebo | | | | | | | | |

In the cumulative responder analysis 61.93% of improvement was observed in the treatment group while it was 2.7% improvement in placebo.

TABLE 24
Cumulative responder analysis with percentage
improvement of pain relief for the treatment
group and placebo groups

| Time | AUC | | % Improvement | |
|---|---|---|---|---|
| (hrs) | treatment | Placebo | treatment | Placebo |
| 1 | 0.00 | 0.00 | 0.00 | 0.00 |
| 2 | 0.00 | 0.00 | 0.00 | 0.00 |
| 3 | 137 | 76 | 1.4 | 0.8 |
| 4 | 618 | 272 | 6 | 2.7 |
| 5 | 2061 | 272 | 21 | 2.7 |
| 6 | 6193 | 272 | 62 | 2.7 |

The mean TOTPAR at 6 h was 198 for the treatment group and 32 for the placebo. There was a statistically significant difference in the pain relief between the treatment group and placebo at 360 min.

TABLE 25
Total pain relief of treatment group and placebo at the end of 6 h.

| Time | Treatment group | Placebo |
|---|---|---|
| (mins) | Mean TOTPAR | Mean TOTPAR |
| 180 | 3 | 6 |
| 360 | 198 | 32 |

The Restricted Mean Survival Time (RMST) of treatment group was 269 minutes compared to 312 minutes in placebo and pain relief was significantly better than placebo.

TABLE 26
Onset of Analgesia-Perceptible Pain Relief (PPR) (minutes)

| | Treatment | Placebo |
|---|---|---|
| | group | |
| Mean PPR | 269 | 312 |
| Restricted Mean Survival Time | | |

The quality of pain assessed by McGill questionnaire was analysed using the Wilcoxon and paired t test. There was a significant difference in the treatment group in the sensory, affective domains and total score of MPQ whereas the placebo group had no significant change. In the VAS and PPI also the treatment group has significant difference whereas the placebo was without significant change.

TABLE 27
MCGILL Quality of Pain Relief Pre-Post analysis
between treatment group and placebo

| | | Mean | Diff |
|---|---|---|---|
| Sensory-Treatment group | Predose | 10 | 4 |
| | Postdosee | 6 | |
| Sensory-Placebo | Predose | 9 | −1 |
| | Postdose | 10 | |
| Affective-Treatment group | Predose | 2 | 1 |
| | Postdose | 1 | |
| Affective-Placebo | Predose | 2 | 0 |
| | Postdose | 2 | |
| Total score MPQ Response- | Predose | 11 | 4 |
| Treatment group | Postdose | 7 | |
| Total score MPQ Response- | Predose | 12 | 0 |
| Placebo | Postdose | 12 | |
| VAS (mm) Treatment group | Predose | 64 | 20 |
| | Postdose | 44 | |
| VAS (mm)-Placebo | Predose | 68 | −2 |
| | Postdose | 70 | |
| PPI-Treatment group | Predose | 3 | 2 |
| | Postdose | 1 | |
| PPI-Placebo | Predose | 3 | 0 |
| | Postdose | 3 | |

In conclusion, pain relief formulation (treatment group) establishes the fast onset of pain relief action in participants with acute musculoskeletal pain. Formulation at the dosage of 500 mg significantly improved pain intensity and perceptible pain relief was achieved as early as four hours of treatment.

### Example 7

Efficacy of Fast Pain Relief Formulation in Menstrual Cramp Pain Associated with Primary Dysmenorrhea.

This is a randomized, double-blind, placebo-controlled study in adolescent and adult female participants with primary dysmenorrhea of at least moderate pain. 60 qualified participants were randomized in two groups in 1:1 ratio to a single dose of fast pain relief formulation or placebo (two soft gel of 500 mg). Before dosing, the participants were completed a baseline (immediately before dosing) 0-10 NRS menstrual cramp pain intensity evaluation.

After taking the study medication, the participant completed the pain/pain relief assessment (0-10 NRS and the 0-4 categorical pain relief scale) at 0.5, 1, 1.5, 2, 2.5, 3, 3.5, 4, 4.5, 5-, 5.5- and 6-hours post-dose.

At 6 hours post-dose, the participant completed the Global Evaluation of her overall satisfaction with the effectiveness of the study medication.

The NRS is a 11-point scale in which 0 represents ‘no pain’ and 10 represents the worst pain possible. For 0-10 NRS scales, the rating chosen has a specific meaning in terms of the impact of pain on functioning. In most samples, ratings in the 1-4 range have a minimal impact on pain, and can be viewed as representing “mild” pain. Once ratings reach 5 or 6, these ratings can be viewed as “moderate” pain. Ratings ranging from 7-10 have the greatest impact on functioning and can be viewed as representing “severe pain”.

The pain relief scale is a categorical scale having a positive progression from ‘No relief’ to ‘Complete relief’.

| Code | Category |
|---|---|
| 0 | No Relief |
| 1 | A Little Relief |
| 2 | Some Relief |
| 3 | A Lot of Relief |
| 4 | Complete Relief |

A question that rates the drug as a pain reliever at 6 hours post-dose or immediately at the first intake of rescue medication.

| Code | Category |
|---|---|
| 0 | Poor |
| 1 | Fair |
| 2 | Good |
| 3 | Very good |
| 4 | Excellent |

The primary outcome measure is Total pain relief scores (TOTPAR) using pain relief categorical scale (0-4). The secondary outcome measures are Summed pain intensity difference (SPID) over the 6-hour study period (SPID0-6) using 0-10 NRS, Pain intensity difference (PID) score and Global evaluation at 6 hrs post dose.

TABLE 28
Pain Intensity Difference (PID) from Baseline with comparison to

| | | Change from | | Change from |
|---|---|---|---|---|
| Time (Hrs) | Placebo | baseline | Treatment | baseline |
| 0 | 7.23 | | 7.23 | |
| 1 | 6.9 | 0.33 | 3.5 | 3.8 |
| 2 | 7.03 | 0.2 | 1.96 | 5.3 |
| 3 | 7.03 | 0.2 | 1.06 | 6.2 |
| 4 | 6.9 | 0.33 | 0.6 | 6.6 |
| 5 | 6.9 | 0.3 | 0.47 | 6.76 |
| 6 | 6.96 | 0.27 | 0.33 | 6.9 |

SPID was calculated as the sum of the differences between the pain scores and baseline pain score over a period of time. Sum of pain intensity difference (SPID6) showed a significant difference at 6 hr when compared to placebo.

TABLE 29
SPID(0-6)

| Treatment | Placebo |
|---|---|
| 34 | 2 |

TOTPAR was calculated using data from a 5-point categorical pain relief scale (none=0, slight=1, moderate=2, good=3, or complete pain relief=4) as the sum of pain relief scores over a period of time.

TABLE 30
Total pain relief of treatment group and placebo at the end of 6 h.

| Time | Treatment group | Placebo |
|---|---|---|
| (mins) | Mean TOTPAR | Mean TOTPAR |
| 360 | 19 | 2 |

The mean TOTPAR at 6 hours was 19 for the treatment group and 2 for the placebo. There was a statistically significant difference in pain relief between the treatment group and placebo at 6 hrs.

Percentage Max TOTPAR and percentage Max SPID were calculated for each individual and categorised as <30%, 30-49%, 49-69% and >70%. Maximum TOTPAR for an individual is the maximum relief score obtained multiplied by time in hours. Maximum possible SPID for an individual is the initial pain rating multiplied by number of hours over which ratings were recorded. Values for TOTPAR and SPID of each individual were respectively converted into percentage of maximum TOTPAR and percentage of maximum SPID by dividing with calculated maximum value of TOTPAR and SPID.

80% of participants in the treatment group are in the TOTPAR percentage category of ≥70% max TOTPAR. In the placebo group, 66.67% of participants were having <30% of max TOTPAR.

TABLE 31
Responder profile for % MAX TOTPAR

| Category | | | | |
|---|---|---|---|---|
| (% Max | | Treatment | Placebo | Treatment |
| TOTPAR) | Placebo | group | (%) | (%) |
| <30 | 20 | 0 | 66.67 | 0 |
| 30-49 | 4 | 0 | 13.33 | 0 |
| 50-69 | 1 | 6 | 3.33 | 20 |
| >=70 | 5 | 24 | 16.67 | 80 |

Six participants in placebo group and 30 participants in treatment group was having ≥50% max TOTPAR and obtained NNT was 1.25 for treatment group compared to placebo, also signifies the effectiveness of treatment.

76.67% of participants in the treatment group was having ≥70% max SPID whereas in the placebo group, 100% participants were having <30% of max SPID.

TABLE 32
Responders profile for % MAX SPID

| | | Treatment | Placebo | Treatment |
|---|---|---|---|---|
| Category | Placebo | group | (%) | (%) |
| <30 | 30 | 0 | 100 | 0 |
| 30-49 | 0 | 1 | 0 | 3.33 |
| 50-69 | 0 | 6 | 0 | 20 |
| >=70 | 0 | 23 | 0 | 76.67 |

### Global Evaluation

### Effectiveness of Study Medication

0=Poor
1=Fair
2=Good
3=Very good
4=Excellent

TABLE 33
Global Evaluation

| | | Poor | Fair | Good | Very good | Excellent |
|---|---|---|---|---|---|---|
| | | (=0) | (=1) | (=2) | (=3) | (=4) |
| Placebo | Count | 25 | 5 | 0 | 0 | 0 |
| | % | 83.3% | 16.7% | 0 | 0% | 0% |
| Test | Count | 0 | 0 | 0 | 8 | 22 |
| formulation | % | 0% | 0% | 0 | 26.7% | 73.3% |

In the treatment group, 73.3% reported that the formulation is an excellent pain reliever and 26.7% reported the formulation is very good for pain relief. Whereas in the placebo group, 83.3% reported poor pain relief.

### Example 8

A randomized, placebo-controlled, study was conducted for analysing the efficacy of a formulation of Turmeric and Boswellia extracts in sesame oil (treatment group) compared to Turmeric and Boswellia extract powder (extract powder) for exercise-induced acute musculoskeletal pain. For this study, 150 healthy male and female subjects with exercise-induced acute musculoskeletal pain with a pain intensity of 5 or above in the numerical rating scale (NRS) were selected. Participants were divided into three groups with 50 subjects in each group. Group 1 received a single dose of 2×500 mg capsule of Turmeric and Boswellia extracts in sesame oil. Group 2 received a single dose of 2×500 mg capsule of Turmeric and Boswellia extract powder. Group 3 subjects were administered with 2×500 mg of capsule of placebo. The treatment duration was 6 hours. The primary outcome in this study was a change in the sum of pain intensity difference (SPID) taken at resting condition. SPID is derived from the NRS scale which is a 11-point scale in which 0 represents ‘no pain’ and 10 represents the worst pain possible. The NRS will be taken at baseline and at each 30 minutes interval post dose for up to 6 hours at rest, on movement and on application of pressure at the affected part. The secondary outcome variables included total pain relief (TOTPAR) measured using a categorical scale called Pain Relief Scale (PRS) which has a positive progression from No Relief to Complete Relief (scored 0-4). The PRS response was also taken at intervals of 30 min. After dosing the onset of analgesia was measured using double-stopwatch method.

TABLE 34
Analysis of sum of pain intensity differences at 6 hours (SPID6)
at rest, movement and pressure.

| SPID6 | Placebo | Treatment group | Extract powder |
|---|---|---|---|
| rest | 1.8 | 31 | 8 |
| move | 1.7 | 34 | 10 |
| pressure | 2 | 34 | 9 |

The sum of pain intensity differences at 6 h showed that treatment group had significant decrease in pain intensity at rest, pressure and movement (SPID6rest, SPID6pres, SPID6mov) compared to extract powder and Placebo.

TABLE 35
Mean of total pain relief (TOTPAR) derived from
the pain relief scale (PRS) at 6 hours (TOTPAR6)
and at rest, movement (move) and pressure

| | Mean TOTPAR | | |
|---|---|---|---|
| | Rest | Move | Pressure |
| Extract powder | 6 | 6 | 6 |
| Placebo | 1 | 2 | 1 |
| Treatment group | 20 | 17 | 20 |

The mean total pain relief after 6 hours in treatment group was significantly better than the other two groups (TOTPAR6rest, TOTPAR6press, TOTPAR6mov). Extract powder was significantly better than placebo (TOTPAR6rest, TOTPAR6pres, TOTPAR6mov). At rest, pressure and movement 98% subjects reported a pain relief of greater than 50% of Max TOTPAR in the treatment group, whereas only 6% reported that in Placebo. The extract powder group had 6% in rest and pressure but only 2% reported greater than 50% Max TOTPAR on movement.

Number needed to treat (NNT) is one way to communicate the effectiveness of a treatment. NNT is the number of patients needed to treat to prevent one additional bad outcome (death, stroke, etc.). A perfect NNT would be 1. where everyone improves with treatment and no one improves with control. A higher NNT indicates that treatment is less effective.

3 participants in placebo group and 49 participants in the treatment group was having ≥50% max TOTPAR. Therefore, the NNT was 1.1 for the treatment group compared to placebo, also signifies the effectiveness of treatment.

TABLE 36
perceptible and meaningful pain relief (PPR & MPR)

| Group | PPR (hrs) | MPR (hrs) |
|---|---|---|
| Extract powder | 3.9 | 5.8 |
| Placebo | 4.2 | 5.9 |
| Treatment group | 1.8 | 2.7 |

The perceptible pain relief was 1 hr 48 min for the treatment group, 3 hr 54 min for extract powder and 4 h 12 min for placebo. Treatment group had a highly significant treatment effect with a PPR difference of 2.2 against extract powder and 2.4 against placebo while extract powder did not have a significant treatment effect against placebo. The meaningful pain relief (MPR) was 2 hr 42 min for the treatment group, 5 hr 48 min for extract powder and 5 h 54 min for placebo. Test formulation had a highly significant treatment effect with a MPR difference of 3.1 against extract powder and 3.2 against placebo while extract powder did not have a significant treatment effect against placebo. Treatment group achieved PPR 2.2 times faster than extract powder and 2.4 times faster than the placebo. Treatment group achieved MPR 2.2 times faster than extract powder and placebo.

The above specification is illustrative and not restrictive. Many variations of the disclosed composition and process will become apparent for a person skilled in the art upon review of the specification.

1. A plant-based formulation for fast pain relief comprising a mixture of solid Turmeric extract and solid Boswellia extract, wherein the mixture is loaded into an oil with a Hydrophilic-lipophilic balance value ranging from 10 to 19 wherein Turmeric extract and Boswellia extract are in about 1:1 to 1:99 ratio by weight, and mixture has a particle size of 200 micrometre to about 100 nanometres.

2. The plant-based formulation as claimed in claim 1, wherein said mixture consists of:
a. the Turmeric extract—10 to 50 wt %;
b. the Boswellia extract—1 to 30 wt %; and
c. a Sesame oil—40 to 90 wt %.

3. The plant-based formulation as claimed in claim 2, wherein said mixture consists of:
a. the Turmeric extract about—30±2 wt %;
b. the Boswellia extract about—10±2 wt %; and
c. the Sesame oil about—60±2 wt %.

4. The plant-based formulation as claimed in claim 1, wherein the mixture is blended with the oil in 1:10 to 1:1 weight ratio.

5. The plant-based formulation as claimed in claim 1, wherein solid Turmeric extract and solid Boswellia extract is having curcuminoids and Acetyl-11-keto-beta-boswellic acid respectively and said curcuminoids in the solid Turmeric extract and Acetyl-11-keto-beta-boswellic acid in the solid Boswellia extract are in 2:1 to 99:1 ratio.

6. The plant-based formulation as claimed in claim 1, wherein the Turmeric extract and Boswellia extract are in about 2.8:1 to 3:1 w/w ratio.

7. The plant-based formulation as claimed in claim 5, wherein the Curcuminoids is in solid Turmeric extract form comprising at least 80% curcuminoids, and Acetyl-11-keto-beta-boswellic acid is in solid Boswellia extract form comprising at least 10% by weight Acetyl-11-keto-beta-boswellic acid.

8. The plant-based formulation as claimed in claim 5, wherein the curcuminoids consist of curcumin, bisdemethoxycurcumin, and demethoxycurcumin, and they are derived from Turmeric rhizome; and Acetyl-11-keto-beta-boswellic acid is derived from Boswellia resin.

9. The plant-based formulation as claimed in claim 1 further comprises one or more additive and is made into a dosage form, wherein the dosage form is selected from a group consisting of softgel capsule, capsule, paste, ointment, infusion, injection, ampoule, solution, suspension, emulsion, cream and oil.

10. The plant-based formulation as claimed in claim 9, wherein the formulation is moulded into a unit dose of 500 to 2000 mg.

11. The plant-based formulation as claimed in claim 1, wherein the oil is selected from group consisting of sesame oil, coconut oil, palm oil, olive oil, sunflower oil, mustard oil, lemon grass oil, bran oil, groundnut oil, lavender oil, flax seed oil, grape seed oil, almond oil, avocado oil, soyabean oil, walnut oil, cashew oil, canola oil, corn oil, safflower oil, cotton seed oil, ramtil oil, niger oil, hemp oil, Turmeric oil, ginger oil, animal fat, medium-chain triglycerides or combination thereof.

12. A method for hastening the onset of analgesia in a mammal experiencing acute musculoskeletal pain by administering the plant-based formulation of claim 1.

13. (canceled)

14. The method as claimed in claim 12, wherein the Musculoskeletal Pain is exercise-induced pain and is selected from a group comprising of low back pain, general whole-body pain, myalgia, headache, neck pain, limb pain, grade one sprain, joint pain, acute soft tissue injury, acute injuries of ligaments and acute injuries of tendons.

15. (canceled)

16. The method as claimed in claim 12, wherein the musculoskeletal pain is exercise-induced and is experienced in a body part selected from Head and neck, Trunk, Upper limb, Lower limb and general musculoskeletal pain.

17. A method of reducing menstrual cramp pain in mammal with primary dysmenorrhea by administering the plant-based formulation of claim 1.

18. A method of preparation of a plant-based formulation for fast pain relief by enhancing oral bioavailability comprising the steps of: a) mixing solid Turmeric extract and solid Boswellia extract in about 2.8:1 to 3:1 w/w ratio; b) the mixture was added to edible oil and mixed well to get a high viscose blend; and c) passing the resultant blend through a bead mill a number of times resulting in an yellow coloured liquid with a viscosity in the range of 270 to 320 mP·s.

19. The method of preparation as claimed in claim 18, wherein the edible oil is selected from group consisting of sesame oil, coconut oil, palm oil, olive oil, sunflower oil, mustard oil, lemon grass oil, bran oil, groundnut oil, lavender oil, flax seed oil, grape seed oil, almond oil, avocado oil, soyabean oil, walnut oil, cashew oil, canola oil, corn oil, safflower oil, cotton seed oil, ramtil oil, niger oil, hemp oil, Turmeric oil, ginger oil, animal fat, medium-chain triglycerides or combination thereof.

20. The method of preparation as claimed in claim 19, wherein the edible oil is sesame oil.

21. The method of preparation as claimed in claim 18, wherein anti-oxidants are dosed at 2000 ppm of the said edible oil as preservatives which are a combination of rosemary and green tea extract.

22. The method of preparation as claimed in claim 18, wherein milling is carried out until the particle size of the mixture is below 200 micrometres and above 100 nanometres.
